SEQUENCE LISTING (10 sequences)

SEQ ID NO: 1 - DNA, 21 bp, Artificial sequence
  Synthesized primer
ggagaaccaagagcagggaga

SEQ ID NO: 2 - DNA, 28 bp, Artificial sequence
  Synthesized primer
aagcttctctttccccgtttcgcctcag

SEQ ID NO: 3 - DNA, 26 bp, Artificial sequence
  Synthesized primer
accggtggcatgaatgacagccggcc

SEQ ID NO: 4 - DNA, 20 bp, Artificial sequence
  Synthesized primer
aggctgaggcaggaggattg

SEQ ID NO: 5 - DNA, 20 bp, Artificial sequence
  Synthesized primer
ctccagtgaggggatccctg

SEQ ID NO: 6 - protein, 19 aa, Homo sapiens
NTRFTITLNYKDPLTGDEE

SEQ ID NO: 7 - protein, 19 aa, Pan troglodytes
NTRFTITLNYKDPLTGDEE

SEQ ID NO: 8 - protein, 19 aa, Bos taurus
DARFAITLNNKDALTGDEE

SEQ ID NO: 9 - protein, 19 aa, Mus musculus
DTRFAITLNNKDALTGDEE

SEQ ID NO: 10 - protein, 19 aa, Rattus norvegicus
DTRFAITLNNKDALTGDEE

(19) United States
(12) Patent Application Publication
(10) Pub. No.: US 2013/0345074 A1
(43) Pub. Date: Dec. 26, 2013

(54) Biomarker and method for evaluating risk for Parkinson's disease

(71) Applicant: NATIONAL TAIWAN NORMAL UNIVERSITY, Taipei City (TW)

(72) Inventors: Guey-Jen LEE-CHEN, Taipei City (TW); Chiung-Mei CHEN, Taipei City (TW); Yih-Ru WU, Taipei City (TW)

(21) Appl. No.: 13/803,508

(22) Filed: Mar. 14, 2013

(30) Foreign Application Priority Data
Jun. 20, 2012 (TW) 101122038

(51) Int. Cl. C12Q 1/68; C12N 9/00; G01N 33/68

(52) U.S. Cl. CPC C12Q 1/6883; G01N 33/6896; C12N 9/93
USPC 506/9; 435/6.11; 435/7.4; 435/4; 536/23.2; 435/183

(57) ABSTRACT

A biomarker and a method for evaluating a risk for Parkinson's disease are disclosed. The method comprises: obtaining a sample from a tester, analyzing the polymorphic biomarker of the sample, wherein the biomarker is substrate-specifying subunit of SCF E3 ubiquitin ligase complex-FBXO7 gene; and when the cDNA sequence in position 155 of the biomarker is G or the amino acid sequence in position 52 of the biomarker is cysteine, it represents that the tester has a lower risk for Parkinson's disease.

### CROSS REFERENCE TO RELATED APPLICATION

This application claims the benefits of the Taiwan Patent Application Serial Number 101122038, filed on Jun. 20, 2012, the subject matter of which is incorporated herein by reference.

### BACKGROUND OF THE INVENTION

1. Field of the Invention

The present invention relates to a biomarker and a method for evaluating a risk for Parkinson's disease. More specifically, the present invention relates to a method using the amino acid sequence in position 52 of substrate-specifying subunit of SCF E3 ubiquitin ligase complex-FBXO7 as a biomarker.

2. Description of Related Art

Parkinson's disease (PD) is a heterogeneous group of common neurodegenerative disorder with multifactorial etiology as well as a slowly progressive disorder that affects movement, muscle control, and balance. Symptoms of Parkinson's disease usually manifest gradually and affect people around the age of 50-60. Part of the disease process develops as cells are destroyed in certain parts of the brain stem, particularly the crescent-shaped cell mass known as the substantia nigra. Neurons in the substantia nigra send out fibers to tissue located in both sides of the brain and the neurons release essential neurotransmitters that help control movement and coordination.

The cause of the disease currently remains unknown for 90% of the patients, only about 10% of the patients with PD are induced by gene mutation and environmental factors. Although PD could not be cured, the symptoms of PD can be reduced through drug treatment, surgery, or other auxiliary treatments so the patients can obtain the better quality of life.

Over the past 15 years, molecular genetic studies have validated the link between eight genes and rare dominant or recessive monogenic forms of PD: SNCA, Parkin, PINK1, DJ-1, LRRK2, ATP13A2, VPS35 and EIF4G1. The functional studies on their protein products and the pathogenetic effects related to their mutations suggest that oxidative stress damage, mitochondrial dysfunction, accumulation of aberrant or misfolded proteins, and failure of cellular clearance systems greatly contribute to the pathogenesis of PD. Although mutations in these genes have also been found in some of late onset sporadic PD, the primary cause of the majority of PD remains unknown.

Therefore, it is desirable to provide a method for evaluating a risk for Parkinson's disease owing to the increased population thereof, in hope that PD progression can be delayed through early evaluation of the risk for Parkinson's disease so as to assist the treatment strategies. Finally, the patients of Parkinson's disease can obtain the better quality of life.

### SUMMARY OF THE INVENTION

The object of the present invention is to provide a method for evaluating a risk for Parkinson's disease, so as to examine the risk for a tester to contract PD.

Another object of the present invention is to provide a biomarker for evaluating a risk for Parkinson's disease, so as to judge the level of the risk for PD of a tester.

To achieve the aforementioned object, the present invention provides a method for evaluating a risk for Parkinson's disease, comprising the following steps: (A) obtaining a nucleic acid-contained sample from a tester; and (B) analyzing a biomarker of the sample, wherein the biomarker is a substrate-specifying subunit of SCF E3 ubiquitin ligase complex-FBXO7 gene; and when the cDNA sequence in position 155 of the biomarker is G or the amino acid sequence in position 52 of the biomarker is cysteine, it represents that the tester has a lower risk for Parkinson's disease.

In the step (B), any method known for DNA, RNA, or cDNA analysis in the art can be used to analyze the nucleic acid-contained sample without limitation. For example, polymerase chain reaction (PCR), quantitative real-time reverse transcription PCR, reverse transcription PCR, gel electrophoresis, single nucleotide polymorphism (SNP) microarray, or restriction fragment length polymorphism (RFLP). Preferably, PCR or RFLP is used to detect the expression of the biomarker of the nucleic acid-contained sample.

In the method of the present invention, the biomarker including the cDNA sequence in position 155 of FBXO7 can be used without limitation. Preferably, the biomarker is a nucleotides sequence, a complementary strand of the nucleotides sequence, a derivative of the nucleotides sequence, or a fragment of the nucleotides sequence of FBXO7; or a combination thereof.

Besides, the present invention provides another method for evaluating a risk for Parkinson's disease, comprising obtaining a protein sample from a tester; and analyzing a biomarker of the sample, wherein the biomarker is substrate-specifying subunit of SCF E3 ubiquitin ligase complex-FBXO7 protein; and when the expression of FBXO7 protein is high, it represents that the tester has a lower risk for Parkinson's disease.

In the aforementioned step, any method known for protein analysis in the art can be used to analyze the protein sample without limitation. For example, western blot analysis (WESTERN), enzyme-linked immunosorbent assay (ELISA), immunohistochemistry (IHC), immunoprecipitation (IP) or mass spectrometry (MS). Preferably, western blot analysis is used to detect the expression of the biomarker of the protein sample.

Herein, the biomarker including the amino acid sequence in position 52 of FBXO7 can be used without limitation. Preferably, the biomarker is proteins, protein derivatives, peptide fragments of proteins, or mutation proteins of FBXO7; or a combination thereof.

In two aforementioned methods for evaluating a risk for Parkinson's disease, the sample can be collected from blood, formalin-fixed tissue, hair, urine, saliva, or nucleic acid-contained tissue from the tester. In other words, the sample can be genomic DNA, RNA, or proteins of the tester.

In addition, the present invention further provides a biomarker for evaluating a risk for Parkinson's disease, which is a nucleotides sequence, a complementary strand of the nucleotides sequence, a derivative of the nucleotides sequence, a protein sequence, a derivative of the protein sequence, a fragment of the protein sequence, a mutation of the protein sequence, an antibody corresponding to the protein sequence, or a combination thereof at amino acid position 52 of substrate-specifying subunit of SCF E3 ubiquitin ligase complex-FBXO7 gene.

When a cDNA sequence in position 155 of the biomarker of a sample from a tester is G, or an amino acid sequence in position 52 of the biomarker of a sample from a tester is cysteine; it represents that the tester has a lower risk for Parkinson's disease.

F-box protein 7 (FBXO7) mutations have been identified in several families with early-onset parkinsonism with pyramidal tract signs. For instance, homozygous R378G missense mutation in the gene encoding the FBXO7 gene was proposed as the likely disease-causing variant for the familiar akinetic-rigid parkinsonism in an Iranian kindred; homozygous nonsense mutation (R498X) in an Italian family and compound heterozygous mutations (IVS7+1G/T and T22M) in a Dutch family showing unambiguously that recessive FBXO7 mutations cause a neurodegenerative disease with early-onset, parkinsonian-pyramidal phenotype; and the pathogenic R498X mutation was found in one Pakistan family and one Turkey family with complex parkinsonism. Nevertheless, no pathogenetic mutations in the FBXO7 gene were detected on Chinese early-onset parkinsonism patients.

FBXO7 is a member of the F-box-containing protein (FBP) family. Through the interaction between the F-box and the Skp1 protein, FBPs become part of SCF (Skp1-Cullin1-F-box protein) ubiquitin ligase complexes, and play roles in ubiquitin-mediated proteasomal degradation. Additionally, FBPs might also function through non-SCF mechanisms, such as regulating mitochondrial morphology and repressing recombination. FBXO7 mediates ubiquitin conjugation to cIAP1 (an apoptosis inhibitor possessing ubiquitin ligase activity) and TRAF2 (a member of the TNF receptor associated factor protein family with ubiquitin ligase activity), resulting in decreased receptor-interacting protein 1 (RIP1) ubiquitination and lowered NF-κB signaling activity.

Furthermore, a housekeeping gene used in the method of the present invention can be β-actin, tubulin, histone, or glyceraldehyde-3-phosphate dehydrogenase (GADPH). Preferably, the housekeeping gene is tubulin, histone, or glyceraldehyde-3-phosphate dehydrogenase.

Therefore, PD progression can be delayed and the patients of Parkinson's disease can obtain the better quality of life through evaluating the risk for Parkinson's disease to assist the treatment strategies by using the method and the biomarker for evaluating a risk for Parkinson's disease of the present invention.

Other objects, advantages, and novel features of the invention will become more apparent from the following detailed description when taken in conjunction with the accompanying drawings.

### BRIEF DESCRIPTION OF THE DRAWINGS

FIG. 1A is chromatograms of direct cDNA sequencing of Y52C;

FIG. 1B is an experimental data from restriction analysis of Y52C;

FIG. 1C is an evolutionary conservation of the region of FBXO7 Y52C;

FIG. 2A is pictures from confocal microscopy examination of FBXO7-EGFP protein;

FIG. 2B is an experimental data of expression of FBXO7-EGFP fusion proteins;

FIG. 2C is an experimental data of expression of FBXO7-EGFP fusion proteins with cycloheximide treatment;

FIG. 2D is a quantification of FIG. 2C;

FIG. 3 is homology models of wild-type FBXO7 and Y52C;

FIG. 4A is an experimental data of expression and quantification of FBXO7 in HEK-293 cells tranfected with wild type (WT) or Y52C

FBXO7 construct;

FIG. 4B is an experimental data of expression and quantification of TRAF2 in HEK-293 cells tranfected with wild type (WT) or Y52C FBXO7 construct;

FIG. 4C is an experimental data of expression of controls (actin and neomycin) in cells tranfected with wild type (WT) or Y52C FBXO7 construct;

FIG. 5A is an experimental data of expression and quantification of FBXO7-EGFP and TRAF2 in SH-SY5Y cells with doxycycline induction (+Dox) or not (−Dox); and

FIG. 5B is representative microscopic images of neuronal differentiated wild type and Y52C cells (for 21 days) and quantification of neuronal total outgrowth.

### DETAILED DESCRIPTION OF THE PREFERRED EMBODIMENT

A total of 516 unrelated Taiwanese PD subjects (45.0% females) were recruited from the neurology clinics of Chang Gung Memorial Hospital (CGMH). All patients were diagnosed with probable idiopathic PD by two neurologists specialized in movement disorders (Y.-R. Wu and C.-M. Chen). Subjects with prior history of multiple cerebrovascular events or other causes of parkinsonian symptoms (e.g. brain injury or tumor, encephalitis, antipsychotic medication) were excluded. The mean age at onset (AAO) of PD was 62.0±11.5 years, ranging between 19 and 93 years. A group of 516 normal controls without neurodegenerative diseases were recruited from the same ethnic community. Control subjects (50.2% females) had mean age at examination of 60.9±12.3 years, ranging between 20 and 92 years. All examinations were performed after obtaining written informed consent from patients and control individuals.

### [Gene Analysis]

Genomic DNA was extracted from peripheral blood lymphocytes using the standard protocols. For PD patients with onset ≧50 (n=80, mean age at onset 43.7±0.7 years, 33.7% females), RNA was extracted using PAXgene Blood RNA Kit (PreAnalytiX). The RNA was DNase (Stratagene) treated, quantified, and reverse-transcribed to cDNA using High Capacity cDNA Reverse Transcription Kit (Applied Biosystems).

Using polymerase chain reaction (PCR) with designed primers and conditions (Table 1), the 1955-bp amplified FBXO7 cDNA was gel purified and sequenced directly using the ABI PRISM 3130 Genetic Analyzer (Applied Biosystems).

TABLE 1

| | | Product/ |
|---|---|---|
| | Anneal | RFLP |
| | (° C.)/ | enzyme |
| | MgCl₂ | (fragment, |
| Test (amplified region) | (mM) | bp) |
| cDNA sequencing | | |
| F: CTCTTTCCCCGTTTCGCC | 58/1.5 | 1955 |
| (residues 7-24 of | | |
| SEQ ID NO: 2) | | |
| R: GGAGAACCAAGAGCAGGGAGA | | |
| (SEQ ID NO: 1) | | |
| pEGFP-N1-FBXO7 cDNA cloning | | |
| F: AAGCTTCTCTTTCCCCGTTTCGCCTCAG | 62/1.5 | 1727 |
| (SEQ ID NO: 2) | | |
| R: ACCGGTGGCATGAATGACAGCCGGCC | | |
| (SEQ ID NO: 3) | | |
| Y52C (TAC/TGC) | | |
| F: AGGCTGAGGCAGGAGGAT TG | 58/1.5 | PstI^{a}: |
| (SEQ ID NO: 4) | | CTGCAG |
| R: CTCCAGTGAGGGGATCCCTG | | (240/222, |
| (SEQ ID NO: 5) | | 18) |

aThe PstI restriction site was created by PCR using a mismatch primer. For Y52C amplification, the underlines in the primer sequence and enzyme recognition site indicate the mismatch nucleotide and polymorphic site, respectively. For cDNA cloning, the underlines in the primer sequence indicate the introduced HindIII and AgeI restriction sites.

The Y52C variants were verified by genomic DNA PCR and sequencing. For population screening, the Y52C was examined using the PstI restriction enzyme as shown in Table 1. The digested PCR products were visualized with ethidium bromide after electrophoresis on 2.2% agarose gel.

[FBXO7 cDNA constructs]

Using the designed primers (as shown in Table 1) to remove translation termination codon, the full-length FBXO7 cDNA fragments from an individual heterozygous for Y52C were cloned into pGEM-T Easy vector (Promega) and sequenced. The 1.7 kb HindIII (added in the forward primer)-AgeI (added in the reverse primer) fragment were removed from pGEM-T Easy vector and ligated into the corresponding sites of pEGFP—N1 (Clontech) to generate wild-type and Y52C FBXO7 cDNA in-frame fused to the EGFP gene.

[Cell Cultivation and Transfection]

Human embryonic kidney (HEK)-293 (ATCC No. CRL-1573) cells were cultivated in Dulbecco's modified Eagle's medium containing 10% fetal bovine serum in a 37° C. humidified incubator with a 5% CO₂ atmosphere. Cells were plated into 6-well (6×10⁵/well) dishes, grown for 20 hr and transfected by the lipofection method (GibcoBRL) with the EGFP-tagged FBXO7 constructs (4 μg/well). The cells were grown for another 48 hr. To evaluate the stability of FBXO7 protein, protein synthesis inhibitor cycloheximide (200 μg/ml) was added 24 hr after transfection for 0, 6, 12, 24, 36, and 48 hr before protein preparation.

### [Confocal Microscopy Examination]

For visualizing intracellular FBXO7-EGFP protein, transfected cells on coverslips were stained with 4′-6-diamidino-2-phenylindole (DAPI) to detect nuclei. The stained cells were examined for dual fluorescent imaging using a Leica TCS confocal laser scanning microscope.

[Protein Preparation]

For total protein preparation, cells were lysed in hypotonic buffer (20 mM HEPES (pH 7.4), 1 mM MgCl₂, 10 mM KCl, 1 mM DTT, and 1 mM EDTA (pH 8.0)) containing the protease inhibitor mixture (Sigma). After sonication and sitting on ice for 20 min, the lysates were centrifuged at 14,000×g for 30 min at 4° C. Protein concentrations were determined using the Bio-Rad protein assay kit and albumin referred as standards.

### [Western Blot Analysis]

Total proteins (25 μg) were electrophoresed on 10% SDS-polyacrylamide gel and transferred onto nitrocellulose membrane (Schleicher and Schuell) by reverse electrophoresis. After being blocked, the membrane was stained with anti-FBXO7 (1:3000 dilution, Abnova), anti-TRAF2 (1:500 dilution, Santa cruz), anti-tubulin (1:10000 dilution, GeneTex), anti-neomycin (1:1000 dilution, Millipore), anti-GAPDH (1:1000 dilution, MDBio), or anti-actin (1:10000 dilution, Millipore) antibody. The immune complexes were detected using horseradish peroxidase-conjugated goat anti-mouse (Jackson ImmunoResearch) or goat anti-rabbit (Rochland) IgG antibody (1:10000 dilution) and Immobilon™ Western Chemiluminescent HRP substrate (Millipore).

[FBXO7 SH-SY5Y Cell Lines Generation]

The SH-SY5Y-derived Flp-In host cells and Flp-In™ T-REx™ System (Invitrogen) was used to generate stably induced SH-SY5Y cell lines exhibiting tetracycline-inducible expression of wild-type and Y52C FBXO7. Briefly, the SH-SY5Y host cells were co-transfected with pOG44 plasmid (constitutively expressed the Flp recombinase) and pcDNA5/FRT/TO-FBXO7-EGFP plasmid according to the supplier's instructions. These cell lines were grown in medium containing 5 μg/ml blasticidin and 100 μg/ml hygromycin. Doxycycline (dox, 5 μg/ml) was added to induce EGFP-tagged FBXO7 expression for two days. The proteins were prepared for Western blotting using antibody to FBXO7 or actin as described. Neuronal phenotypes were examined after induced differentiation with retinoid acid (10 μM) and induced expression of FBXO7 for 7 to 21 days.

### [Statistical Analysis]

The genotype frequency data and the expected genotypic frequency under random mating were computed and Chi-square tested for Hardy-Weinberg equilibrium using standardized formula. The genotype and allele association analysis was carried out using the Chi-square test. Odds ratios with 95% confidence intervals (95% CI) were calculated to test association between genotype/allele and disease. Differences in functional assays were analyzed by two tailed Student's t-test. The values of P<0.05 were considered significant.

### [Homology Modeling]

We modeled the three dimensional structures of the wild type and Y52C FBXO7 proteins by comparative methods and energy minimization using the program SWISS-MODEL. The 2.9-Å coordinate set for the crystal structure of human UBC protein (PDB code 2ZVO, chain A) served as the template for modeling the residue 1-79 of human FBXO7. The energy computation was done with the GROMOS96 implementation of Swiss-PdbViewer. The resulting FBXO7 three-dimensional models were manipulated and rendered in PyMOL (The PyMOL Molecular Graphics System, Version 1.2r3pre, Schrödinger, LLC).

### [Results]

### [Mutation/Variant Analysis of FBXO7]

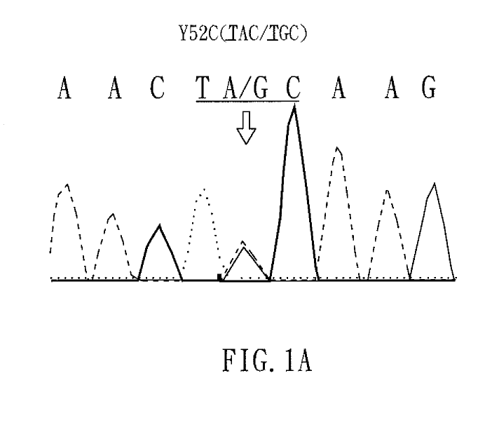

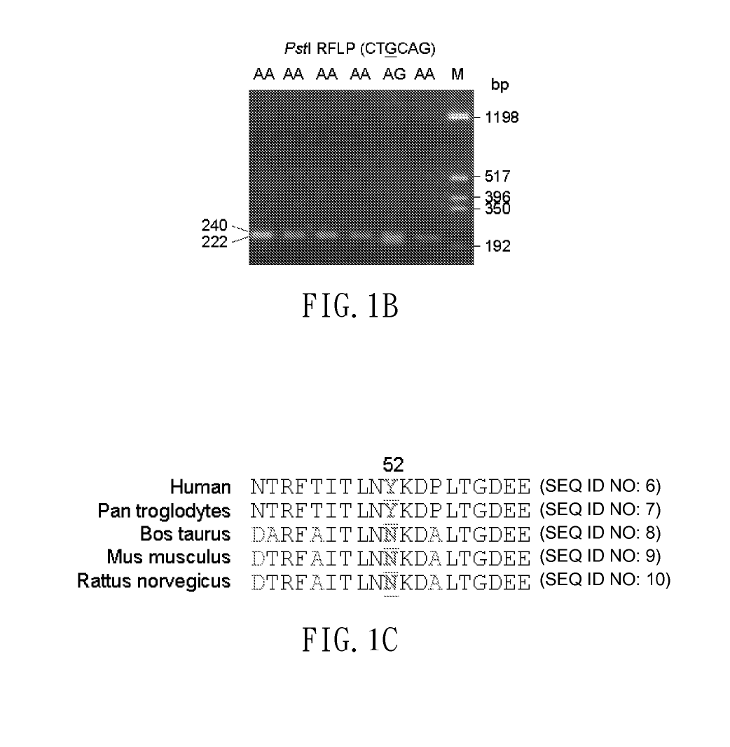

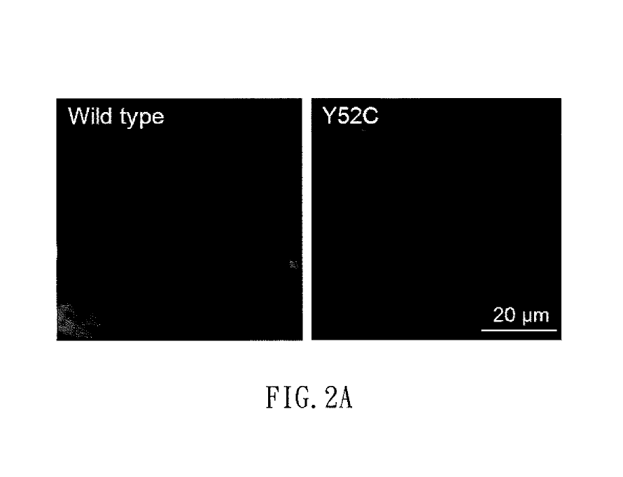

With reference to FIG. 1A, there is shown the chromatograms of direct cDNA sequencing of Y52C. Substitution that caused change in the peptide sequence was identified: a A155G substitution leading to an amino acid change from tyrosine to cysteine in position 52. The variant was confirmed using PCR-restriction fragment length polymorphism (RFLP) method as shown in FIG. 1B. According to FIG. 1C, Y52C is not evolutionary conserved in the known mammalian homologues of the FBXO7 protein.

[Case-control study of Y52C]

A case-control study in a cohort of PD patients (n=516) and ethnically matched controls (n=516) was conducted to assess the association of Y52C with the risk of PD. The genotype and allele distributions of the SNP in patients and controls are displayed in Table 2.

TABLE 2

| | | | |
|---|---|---|---|
| No. (%) | | | |
| PD | Control | Odds ratio^{a} (95% CI) | P-Value |

Genotype/allele
Y52C (TAC/TGC)

| | | | | | | |
|---|---|---|---|---|---|---|
| AA | 512 | (99.2) | 504 | (97.7) | 1.00 | |
| AG | 4 | (0.8) | 12 | (2.3) | 0.33 (0.09-0.95) | 0.055 |
| A | 1028 | (99.6) | 1020 | (98.8) | 1.00 | |
| G | 4 | (0.4) | 12 | (1.2) | 0.33 (0.09-0.95) | 0.056 |

Y52C Combined (Taiwan + China^{b})

| | | | | | | |
|---|---|---|---|---|---|---|
| AA | 645 | (99.1) | 696 | (97.2) | 1.00 | |
| AG | 6 | (0.9) | 20 | (2.8) | 0.32 (0.12-077) | 0.016 |
| A | 1296 | (99.5) | 1412 | (98.6) | 1.00 | |
| G | 6 | (0.5) | 20 | (1.4) | 0.33 (0.12-0.77) | 0.017 |

^{a}Odds ratios were calculated by comparing each value with the major common genotype or allele.
^{b}Luo et al., 2010.

As shown in Table 2, Y52C genotype frequency confirmed to be in the Hardy-Weinberg equilibrium. The frequency of AG genotype (0.8% vs. 2.3%, P=0.046) or G allele (0.4% vs. 1.2%, P=0.046) was significantly lower in PD patients than the controls. When odds ratios of the at-risk genotype/allele were calculated, Y52C AG genotype or G allele demonstrated a trend toward decrease in risk of developing PD (odds ratio: 0.33, 95% confidence interval: 0.09-0.95, P=0.055˜0.056). Meta-analysis combining our patient and control subjects as well as the population in Luo's study yielded results with statistically significant difference in genotype (0.9% vs. 2.8%, P=0.012) and allele (0.5% vs. 1.4%, P=0.012) distribution between patients and controls. The negative association of the Y52C AG genotype or G allele with PD was significant (odds ratio: 0.32-0.33, 95% confidence interval: 0.12-0.77, p=0.016-0.017).

### [FBXO7 Expression Analysis]

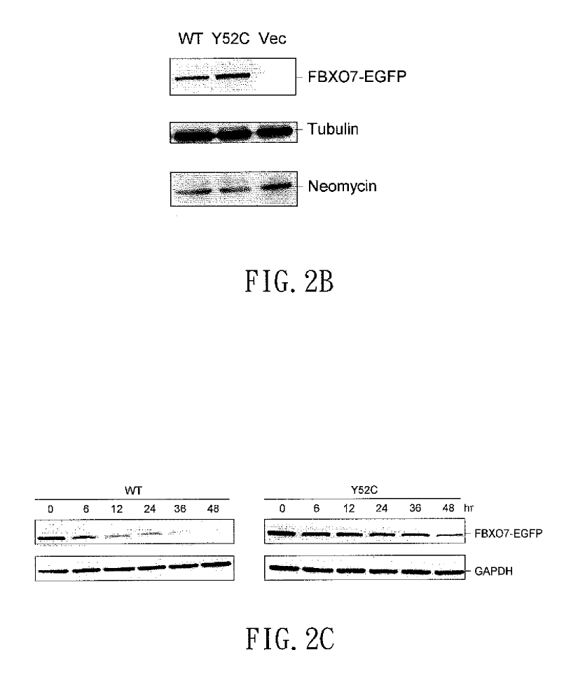

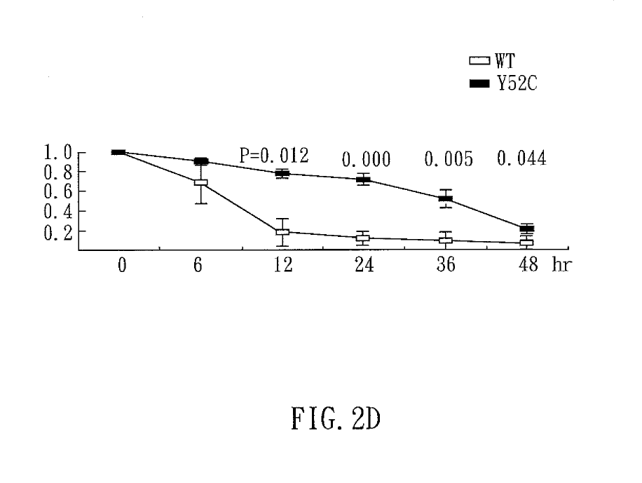

With reference to FIG. 2A, although Y52C FBXO7 protein displayed nuclear and cytosolic staining pattern similar to wild type, a stronger signal was observed with Y52C.

To further examine the transiently expressed FBXO7-EGFP fusion proteins, protein blotting with FBXO7 antibody was performed. As shown in FIG. 2B, while no specific band was detected with vector-transfected cells, FBXO7-EGFP fusion proteins in the expected size range for wild-type and Y52C constructs were observed. However, the protein expression levels of Y52C was increased compared with the wild-type (211%, P=0.016). The stability of Y52C variant was further examined in a cycloheximide (200 μg/ml) chase experiment. While the wild-type protein was degraded to 68%, 18%, 11%, 9% and 7% left after 6, 12, 24, 36, and 48 hr of protein synthesis blocking, reduced rates of decay were observed for Y52C variant (90%, 78%, 72%, 52%, and 21% remained, respectively) (FIG. 2C).

### [Homology Modeling of Y52C]

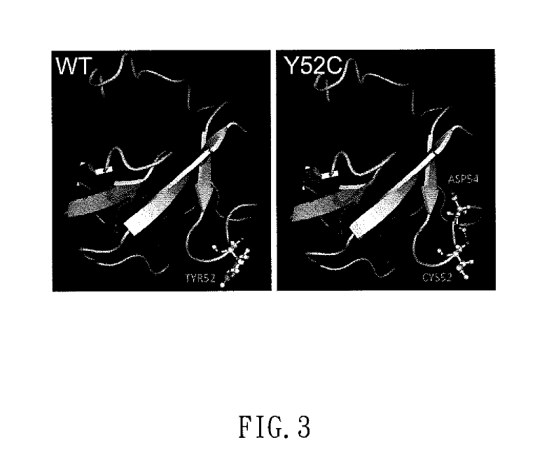

To understand the structure-based information of Y52C polymorphism in FBXO7, homology modeling of wild type and Y52C FBXO7 was performed. After energy minimization, the modeled structures for wild type and Y52C were shown in FIG. 3. The potential energies of wild-type and Y52C variant were −2463.854 and −2471.736 kcal/mol, respectively, indicating Y52 FBXO7 exhibited a more stable feature than wild type. According to hydrogen-bond (H-bond) computing analysis, the H-bond interaction of Tyr54 and Cys52 was shown.

### [FBXO7 Regulating TRAF2 Abundance]

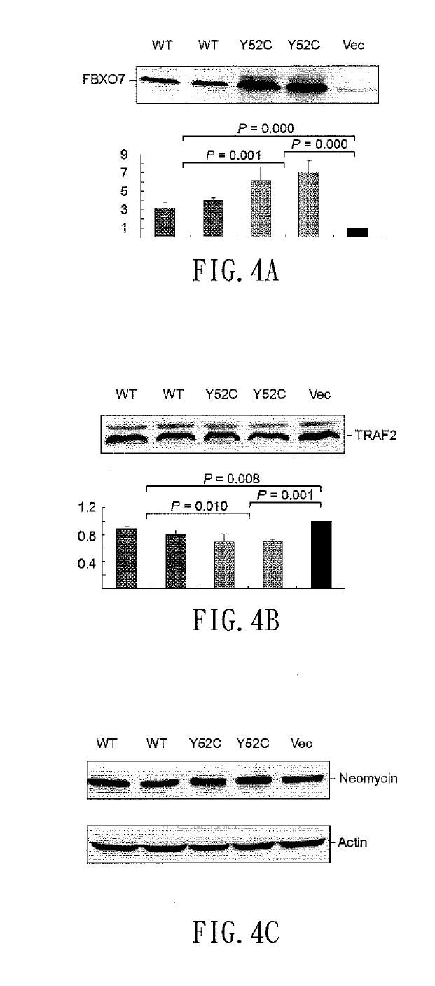

Through binding and mediating ubiquitin conjugation to TRAF2, FBXO7 was identified as a negative regulator of NF-κB signalling. To assess Y52C's effect on TRAF2 abundance, wild-type or Y52C FBXO7 cDNA plasmid was transfected into HEK-293 cells and protein blottings with TRAF2 and FBXO7 antibodies were performed as shown in FIGS. 4A-4B. Referring to FIG. 4A, wild-type or Y52C cDNA transfection significantly increase FBXO7 abundance (3.11˜7.11 folds, P=0.000) while compared with the endogenous FBXO7 level. Between wild-type and Y52C FBXO7, the Y52C level was significantly higher than that of wild-type (6.15˜7.11 vs. 3.11˜4.01, P=0.001). Accompanying that, TRAF2 protein abundance was significantly decreased in wild type or Y52C FBXO7 cells (0.69˜0. 0.89, P=0.008˜0.001) as shown in FIG. 4B. In Y52C cells, the TRAF2 expression level was significantly lower than that of wild-type cells (0.69˜0.70 vs. 0.80˜0.89, P=0.010). In addition, anti-actin and anti-neomycin antibodies were used as loading and transfection controls in FIG. 4C.

### [SH-SY5Y Cell Model]

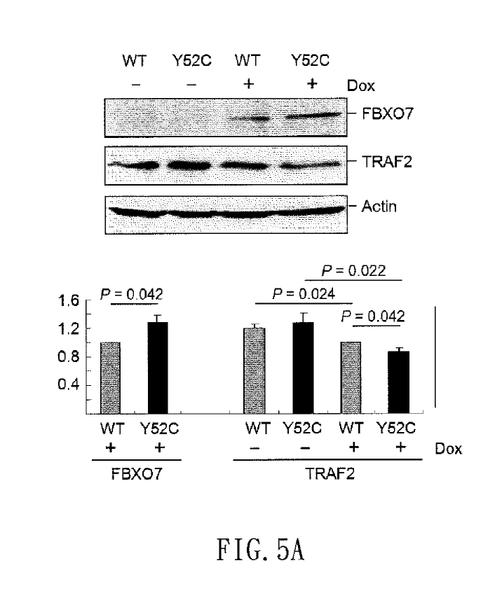

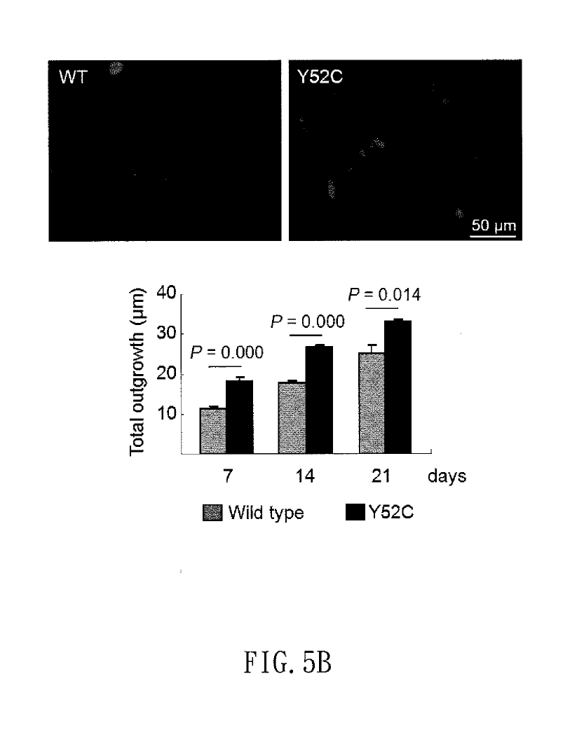

To test the effect of Y52C on neuronal phenotype, we constructed Flp-In SH-SY5Y cells with wild-type or Y52C FBXO7-EGFP expression in an inducible fashion. With regard to FIG. 5A, immunoblot analysis shows that the FBXO7 protein level was significantly increased in Y52C cells as compared to that of wild type cells after induction with doxycycline (+Dox) for 2 days (128%, P=0.042). Compared to the non-induced cells (−Dox), TRAF2 abundance was significantly decreased in both wild type (100% vs. 120%, P=0.024) and Y52C (87% vs. 128%, P=0.022) FBXO7-EGFP expressed cells. The difference of TRAF2 abundance between wild type and Y52C FBXO7-EGFP expressed cells was also significant (100% vs. 87%, P=0.042). These FBXO7 cells were induced for differentiation with retinoic acid for 7 to 21 days. Representative fluorescence microscopy images of cells differentiated for 21 days are shown in FIG. 5B. Significant more total outgrowth in Y52C cells was observed compared to wild type cells after differentiation for 7˜21 days (131˜165%, P=0.014˜0.000).

Although the present invention has been explained in relation to its preferred embodiment, it is to be understood that many other possible modifications and variations can be made without departing from the spirit and scope of the invention as hereinafter claimed.

What is claimed is:

1. A method for evaluating a risk for Parkinson's disease, comprising the following steps:
(A) obtaining a sample from a tester; and
(B) analyzing a biomarker of the sample, wherein the biomarker is substrate-specifying subunit of SCF E3 ubiquitin ligase complex-FBXO7 gene; and when the cDNA sequence in position 155 of the biomarker is G or the amino acid sequence in position 52 of the biomarker is cysteine, it represents that the tester has a lower risk for Parkinson's disease.

2. The method as claimed in claim 1, wherein the sample is collected from blood, formalin-fixed tissue, hair, urine, saliva, or nucleic acid-contained tissue from the tester.

3. The method as claimed in claim 1, wherein the sample is genomic DNA, cDNA, RNA, or protein from the tester.

4. The method as claimed in claim 1, wherein a polymerase chain reaction (PCR), a gel electronphoresis, a single nucleotide polymorphism microarray (SNP microarray), a restriction fragment length polymorphism (RFLP), a western blot analysis, an enzyme-linked immunosorbent assay (ELISA), an immunohistochemistry (IHC), an immunoprecipitation (IP), or a mass spectrometry (MS) is used to analyze the biomarker of the sample in the step (B).

5. The method as claimed in claim 4, wherein the polymerase chain reaction (PCR) and the restriction fragment length polymorphism (RFLP) is used to analyze the biomarker of the sample in the step (B).

6. The method as claimed in claim 1, wherein the biomarker is nucleotides, complementary nucleotides, nucleotide derivatives, nucleotide fragments, proteins, protein derivatives, peptide fragments of proteins, or mutation proteins of FBXO7.

7. The method as claimed in claim 6, wherein the biomarker is nucleotides, complementary nucleotides, nucleotide derivatives, or nucleotide fragments of FBXO7.

8. A biomarker for evaluating a risk for Parkinson's disease, which is a nucleotides sequence, a complementary strand of the nucleotides sequence, a derivative of the nucleotides sequence, a protein sequence, a derivative of the protein sequence, a fragment of the protein sequence, a mutation of the protein sequence, an antibody corresponding to the protein sequence, or a combination thereof at amino acid position 52 of substrate-specifying subunit of SCF E3 ubiquitin ligase complex-FBXO7 gene.

9. The biomarker as claimed in claim 8, wherein when a cDNA sequence in position 155 of the biomarker of a sample from a tester is G, it represents that the tester has a lower risk for Parkinson's disease.

10. The biomarker as claimed in claim 8, wherein when an amino acid sequence in position 52 of the biomarker of a sample from a tester is cysteine, it represents that the tester has a lower risk for Parkinson's disease.
